(19) United States
(12) Patent Application Publication
(10) Pub. No.: US 2026/0109836 A1
(43) Pub. Date: Apr. 23, 2026

(54) PROCESS FOR PROCESSING POLYMER-CONTAINING MATERIALS

(71) Applicant: PLANTICS HOLDING B.V., ARNHEM (NL)

(72) Inventors: Wridzer Jan Willem BAKKER, ARNHEM (NL); Hans Daniël KNEPPER, ARNHEM (NL); Boris Leo Harrie DUIJF, ARNHEM (NL); Geert Jan NOORDZIJ, ARNHEM (NL)

(21) Appl. No.: 19/118,754

(22) PCT Filed: Oct. 6, 2023
(86) PCT No.: PCT/EP2023/077708

§ 371 Date: Apr. 4, 2025

(30) Foreign Application Priority Data
Oct. 7, 2022 (EP) 22200424.4

(51) Int. Cl. C08J 11/14; C07C 51/377

(52) U.S. Cl. CPC C08J 11/14; C07C 51/377; C08J 2367/04

(57) ABSTRACT

The invention pertains to a process for treating a polymer-containing material, the process including: providing a starting material including a polymer which is a polyester derived from aliphatic polyalcohol with 2-15 carbon atoms that includes at least 70 wt% of polyalcohol with at least 3 hydroxyl groups, and aliphatic polycarboxylic acid with 2-15 carbon atoms that includes at least 70 wt% of tricarboxylic acid, the polyester having an extent of polymerization of at least 0.7, and contacting the starting material at a temperature of at least 80° C. for at most 24 hours with a nucleophile to effect depolymerisation of the polymer, resulting in a polymer with an extent of polymerisation which is reduced with at least 0.1 as compared to the polymer in the starting material, and is at a value in the range of 0.1-0.8, the nucleophile comprising at least one of water, liquid polymer which is the polymerisation product of an aliphatic polyalcohol with 2-15 carbon atoms and an aliphatic polycarboxylic acid with 2-15 carbon atoms.

The present invention pertains to process for processing polymer-containing materials, in particular materials containing a renewable and degradable polyester polymer based on a polyalcohol and a polycarboxylic acid, more specifically, a polyester polymer based on an aliphatic polyalcohol with 2-15 carbon atoms and an aliphatic polycarboxylic acid with 2-15 carbon atoms.

In the art, this polymer has been described for large number of widely varying applications.

WO2012140237 describes a composite material comprising 10-98 wt. % of a bio-based particulate or fibrous filler and at least 2 wt. % of a polyester derived from an aliphatic polyalcohol with 2-15 carbon atoms and a polycarboxylic acid, wherein the polycarboxylic acid comprises at least 10 wt. % of tricarboxylic acid. In particular, the filler may be selected from wood chips, wood flakes, sawdust, pulp, e.g., pulp of (recycled) paper or other fiber pulp, and plant-derived fibers such as cotton, linen, flax, and hemp.

WO2012140239 describes a composite material which comprises the same polymer material as the composite of WO2012140237, but in this case a synthetic filler is used, preferably selected from one or more of ceramic, including glass, in particular glass fibers, polymer, in particular polymer fibers, and carbon, in particular carbon fibers.

WO2012052385 describes the same polymer, in the form of a foam.

The references cited above describe polyesters obtained by polymerising polyalcohols with at least 3 hydroxyl groups, in particular glycerol, with polycarboxylic acids with at least three carboxylic acid groups, in particular glycerol. Especially at high extents of polymerisation the use of these monomers results in the formation of thermosets with high strength and high durability, which makes them suitable for use in products such as furniture, building and construction materials (indoor and outdoor use), and other applications requiring resilience. Examples of compositions of this type are described in WO20220106724 and non-prepublished WO 2022214552.

With renewability and recyclability of materials becoming more and more important, there is need in the art for a method for recycling the polymer-containing materials. It would be particularly preferred if it would be possible to convert the material into something which can be reused in the manufacture of high-value materials in an efficient manner. The present invention provides such a method.

The invention pertains to a process for treating a polymer-containing material comprising the steps of
providing a starting material comprising a polymer which is a polyester derived from aliphatic polyalcohol with 2-15 carbon atoms, the aliphatic polyalcohol comprising at least 70 wt. % of polyalcohol with at least 3 hydroxyl groups, and aliphatic polycarboxylic acid with 2 to 15 carbon atoms, the aliphatic polycarboxylic acid comprising at least 70 wt. % of tricarboxylic acid, the polyester having an extent of polymerization, which is the ratio of the fraction of functional groups that have reacted to the maximum of those functional groups that can react, of at least 0.7,
in a depolymerisation step contacting the starting material at a temperature of at least 80° C. for at most 24 hours with a nucleophile to effect depolymerisation of the polymer, resulting in a polymer with an extent of polymerisation which is reduced with at least 0.1 as compared to the extent of polymerisation of the polymer in the starting material, and is at a value in the range of 0.1-0.8, the nucleophile comprising at least one of water, liquid polymer which is the polymerisation product of an aliphatic polyalcohol with 2-15 carbon atoms the aliphatic polyalcohol and an aliphatic polycarboxylic acid with 2-15 carbon atoms.

It has been found that the method according to the invention makes it possible to reduce the molecular weight of the polymer in an efficient and cost effective manner, while providing a material which can be applied in industry in various manners. In particular, the polymer recovered from the process can be re-used in the manufacture of new polymeric materials while maintaining good product quality.

The present invention enables cradle-to-cradle processing of the thermoset polymers used at high extents of polymerisation of at least 0.7, but in particular at least 0.8, or at least 0.9, or at least 0.95 The process according to the invention makes it possible to convert thermoset polymer to a material with a lower extent of polymerisation, which can be used as a starting material to produce new materials, including new thermoset materials. This is a difference with thermoset polymer materials which are conventionally used, which cannot easily be converted to a repolymerisable product in high efficiency while maintaining product quality.

A particular advantage of the process according to the invention is that the nucleophiles used therein are compounds which do not affect a repolymerisation step. If water is used, it will be removed in a repolymerisation step together with the water generated in the esterification step. If the specified liquid polyester or monomers thereof are used, they will be incorporated into the newly formed polymer. If the composition of the polymer or monomers used as nucleophile is matched to the polymer that is depolymerised, the composition of the final product will not change at all. This is different from the nucleophile used in many prior art depolymerisation processes.

The present invention, specific embodiments thereof, and the advantages associated therewith will be discussed in more detail below.

### The Starting Material

The first step in the method according to the invention is the step of providing a starting material comprising a polymer which is the polymerisation product of an aliphatic polyalcohol with 2-15 carbon atoms and an aliphatic polycarboxylic acid with 2-15 carbon atoms, the polymer in the starting material having an extent of polymerisation of at least 0.55.

Within the context of the present invention, the extent of polymerisation of a polymer is defined as the ratio of the fraction of functional groups that have reacted to the maximum of those functional groups that can react. The extent of polymerization can be determined by way of the acid value (in particular for values below 0.5) or gravimetrically (in particular for values above 0.5).

It will be evident that, in order to determine the extent of polymerization of a polymer derived from an aliphatic polyalcohol and an aliphatic polycarboxylic acid with an unknown extent of polymerization using gravimetric analysis, a sample of the polymer with an unknown extent of polymerization is cured at a temperature from 100 to 220° C. until no more water is lost. The extent of polymerization of the polymer is then 1, allowing one to calculate back the extent of polymerization of the sampled polymer using the water lost during the curing.

### The Polymer

The polymer is a polyester derived from an aliphatic polyalcohol with 2-15 carbon atoms and an aliphatic polycarboxylic acid with 2 to 15 carbon atoms.

Suitable polyalcohol monomers for use in the present invention include aliphatic polyalcohols with 2-15 carbon atoms. The aliphatic polyalcohol does not comprise any aromatic moieties, nitrogen atoms or sulphur atoms. In some embodiments, the aliphatic polyalcohol consists of carbon, oxygen and hydrogen atoms. The aliphatic polyalcohol comprises at least two hydroxyl groups, preferably at least three hydroxyl groups. In general, the number of hydroxyl groups will be 10 or fewer, preferably 8 or fewer, more preferably 6 or fewer. The aliphatic polyalcohol has 2 to 15 carbon atoms, preferably 3 to 10 carbon atoms. Examples of suitable aliphatic polyalcohols are 1,2-propane diol, 1,3-propane diol, 1,2-ethane diol, 1,4-butanene diol, glycerol, sorbitol, xylitol, and mannitol. Glycerol, sorbitol, xylitol, and mannitol are preferred examples of suitable aliphatic polyalcohols. Glycerol is the most preferred example of a suitable aliphatic polyalcohol. One reason for this is that glycerol has a melting point of 20 ° C., which allows easy processing (compared to, e.g., xylitol, sorbitol, and mannitol, which all have melting points above 90 ° C.). Moreover, glycerol is easily accessible and results in polymers having desirable properties. Accordingly, in some embodiments, the aliphatic polyalcohol consists essentially of glycerol. As used herein, “consists essentially of” means that other components (here: other aliphatic polyalcohols) may be present in amounts that do not detrimentally affect the properties of the material.

The aliphatic polyalcohol comprises at least 70 wt. % of polyalcohol with at least 3 hydroxyl groups, in particular at least 80 wt. %, more preferably at least 90 wt. %, most preferably 95 wt. %, calculated on the total amount of aliphatic polyalcohol. In some embodiments, the aliphatic polyalcohol consists essentially of polyalcohol with at least 3 hydroxyl groups. It is preferred for the polyalcohol with at least 3 hydroxyl groups to consist for at least 70 wt. % of glycerol, in particular at least 80 wt. %, more in particular at least 90 wt. % or at least 95 wt. %.

Mixtures of different aliphatic polyalcohols may also be used. The aliphatic polyalcohol may comprise at least 50 mol% of glycerol, sorbitol, xylitol, or mannitol, preferably at least 70 mol%, preferably at least 90 mol%. Preferably, the balance is an aliphatic polyalcohol having 3 to 10 carbon atoms. The polyalcohol preferably comprises at least 70 mol% of glycerol, preferably at least 90 mol%, more preferably at least 95 mol%.

In some embodiments, the aliphatic polyalcohol has a ratio of hydroxyl groups over the number of carbon atoms from 1:4 (i.e., one hydroxyl group per four carbon atoms) to 1:1 (i.e., one hydroxyl group per carbon atom). It is preferable for the ratio of hydroxyl groups over the number of carbon atoms to be from 1:3 to 1:1, more preferably from 1:2 to 1:1, still more preferably from 1:1.5 to 1:1. Compounds wherein the ratio of hydroxyl groups to carbon atoms is 1:1 are considered especially preferred.

Suitable polycarboxylic acid monomers for use in the present invention include aliphatic polycarboxylic acids with 2 to 15 carbon atoms, preferably 3 to 10 carbon atoms, in some embodiments 3 to 6 carbon atoms. The aliphatic polycarboxylic acid does not comprise aromatic moieties, or any nitrogen or sulphur atoms. In some embodiments, the aliphatic polycarboxylic acid consists of carbon, oxygen and hydrogen atoms. The aliphatic polycarboxylic acid comprises at least two carboxylic acid groups, preferably three carboxylic acid groups. In general, the number of carboxylic acid groups will be 10 or fewer, preferably 8 or fewer, more preferably 6 or fewer.

The aliphatic polycarboxylic acid comprises at least 70 wt. % of tricarboxylic acid, calculated on the total amount of aliphatic polycarboxylic acid. The aliphatic polycarboxylic acid may comprise at least 80 wt. % of tricarboxylic acid, more preferably at least 90 wt. %, most preferably 95 wt. %. In some embodiments, the aliphatic polycarboxylic acid consists essentially of tricarboxylic acid, preferably essentially of citric acid.

The aliphatic polycarboxylic acid may be a mixture of acids, such as a mixture of tricarboxylic acid(s) and dicarboxylic acid(s). In some embodiments, the aliphatic polycarboxylic acid comprises a combination of 2-30 wt. %, preferably 5-30 wt. %, in some embodiments 10-30 wt. % dicarboxylic acid, and at least 70 wt. %, more preferably at least 80 wt. %, tricarboxylic acid, calculated on the total amount of aliphatic polycarboxylic acid.

The dicarboxylic acid, if used, may be any dicarboxylic acid which has two carboxylic acid groups and, in general, at most 15 carbon atoms. Examples of suitable dicarboxylic acids include itaconic acid, malic acid, succinic acid, glutaric acid, adipic acid, sebacic acid, oxalic acid, maleic acid, fumaric acid, muconic acid, suberic acid, and azelaic acid. Itaconic acid, succinic acid maleic acid and fumaric acid may be preferred.

The tricarboxylic acid may be any tricarboxylic acid which has three carboxylic acid groups and, in general, at most 15 carbon atoms. Examples include citric acid, isocitric acid, aconitic acid (both cis and trans), and 3-carboxy-cis, cis-muconic acid. The use of citric acid is considered preferred, both for reasons of costs and of availability. Where applicable, acids may also be provided in the form of their anhydrides, e.g. citric acid anhydride. In one embodiment, the tricarboxylic acid consists for at least 70 wt. % of citric acid, in particular for at least 80 wt. %, more in particular at least 90 wt. %, still more in particular at least 95 wt. %.

In one embodiment, the polymer is a polyester derived from an aliphatic polyalcohol with 2-15 carbon atoms and an aliphatic polycarboxylic acid with 2 to 15 carbon atoms, wherein the aliphatic polyalcohol comprises at least 70 wt. % of polyalcohol with at least 3 hydroxyl groups, i more preferably at least 80 wt. %, still more preferably at least 90 wt. %, most preferably 95 wt. %, the aliphatic polyalcohol with at least 3 hydroxyl groups preferably being glycerol, and the aliphatic polycarboxylic acid comprises at least 70 wt. % of tricarboxylic acid, calculated on the total amount of acid, preferably at least 80 wt. %, more preferably at least 90 wt. %, most preferably 95 wt. %, the tricarboxylic acid preferably being citric acid.

In one embodiment of the present invention the polymer is derived from a combination of polyalcohol monomers and polycarboxylic acid monomers,
the polyalcohol monomers preferably being selected from aliphatic polyalcohols with 2-15 carbon atoms with at least three hydroxylgroups, e.g., glycerol, sorbitol, xylitol, and mannitol, in particular glycerol,
the polycarboxylic acid monomers being selected from aliphatic polycarboxylic acids with 3-15 carbon atoms with at least three carboxylic acid groups, e.g., citric acid, isocitric acid, aconitic acid (both cis and trans), and 3-carboxy-cis, cis-muconic acid, in particular citric acid.

In the present invention it is preferred for the ratio between the total number of hydroxy-groups and the total number of carboxylic groups in the system to be in the range of 2:1 to 0.5:1, in particular 1.5:1 to 0.6:1, more in particular 1.25:1 to 0.8:1, still more in particular in the range of 1.1:1 to 0.9:1. If the polyalcohol and the polycarboxylic acid fully consist of tri-ols and tri-acids respectively, this translates to a molar ratio of the these compounds in the range of 2:1 to 0.5:1, in particular 1.5:1 to 0.6:1, more in particular 1.25:1 to 0.8:1, still more in particular in the range of 1.1:1 to 0.9:1. If polyalcohols or polyacids with different numbers of hydroxy groups and carboxylic groups are used, this should be taken into account in determining the relative amounts of the compounds to be used.

Having the ratio between the total number of hydroxy-groups and the total number of carboxylic groups in the system within the stipulated ranges is considered advantageous, because it makes it possible to achieve the highest conversion possible. Achieving a high conversion is considered advantageous as there will be a limited amount of hydroxy or acid functional groups left. These groups are hygroscopic in nature, and can have a detrimental effect on the products applicability in demanding applications where durability of the product is required.

As indicated above, the starting polymer has an extent of polymerisation of at least 0.7. The polymer with an extent of polymerization in this range can be obtained by polymerisation of a combination of polyalcohol monomers and polycarboxylic acid monomers. In general, in a first step a mixture of monomers in the liquid phase may be prepared. Depending on the nature of the compounds this can be done, e.g., by heating a mixture of components to a temperature where the acid will dissolve in the alcohol, in particular in glycerol. Depending on the nature of the compounds this may be, e.g., at a temperature in the range of 20-250° C., e.g., 40-200° C., e.g. 60-200° C., or 90-200° C. In one embodiment, the mixture may be heated and mixed for a period of 1 minute to 2 hours, more specifically 5 minutes to 45 minutes, at a temperature of 80-200° C., in particular 100-200° C., in some embodiments 120-180° C. If so desired a suitable solvent, e.g., water may be present. Preferably the amount of water will be kept limited as its evaporation is energy-consuming. It may be preferred to add at most 30 wt. % water, in particular at most 20 wt. % water.

Optionally a suitable catalyst can be used for the preparation of the polyester. Suitable catalysts for the manufacture of polyester are known in the art. Preferred catalysts are those that do not contain heavy metals. Both basic and acidic catalysts may be used. Both homogeneous catalysts and heterogenous catalysts like e.g. those based on zeolites, modified hydrotalcites or resins based as e.g. amberlite or Nafion, can be used. Useful acidic catalysts include, but are not limited to, hydrochloric acid, hydroiodic acid (also indicated as hydriodic acid) and hydrobromic acid, sulfuric acid (H₂SO₄), nitric acid (HNO₃), chloric acid (HClO₃), boric acid, sodium hypophosphite, perchloric acid (HClO₄) trifluoroacetic acid, p-toluenesulfonic acid, sulfonic acid, and trifluoromethanesulfonic acid. Catalysts such as Ti-butoxide, Sn-octanoate, Zn-acetate and Mn-acetate can also be used, although they may be less preferred.

The monomer mixture will be subjected to a heating step to obtain an extent of polymerisation of at least 0.55, in particular at least 0.6. In general, the mixture will be cured at an internal temperature of 80 to 250° C., in particular 220° C., e.g., for 5 seconds to 24 hours. The curing step generally takes place at an internal temperature of at least 80° C., in particular at least 100° C., more in particular at least 120° C., even more in particular at least 130° C. An internal temperature which is very high results in increased side reactions. It is therefore preferred for the internal temperature not to reach values above 250° C. It may be preferred for the internal temperature to be in the range of 130-220° C., in particular 130-200° C. The internal temperature is measured during curing or immediately after the article is removed from a means for curing, such as an oven or a press.

Curing can be carried out using heating technology known in the art, e.g., in in an oven with an oven temperature from 80° C. up to 450° C. Different types of ovens may be used, including but not limited to belt ovens, convection ovens, microwave ovens, infra-red ovens, hot-air ovens, conventional baking ovens and combinations thereof. Vacuum ovens are also considered attractive. Curing can also be carried out through high-frequency heating. Curing can be done in a single step, or in multiple steps. The curing times range from 5 seconds up to 24 hours, depending on the size and shape of the object, on the internal temperature aimed for, and on the heating system applied. Where microwave heating or high-frequency heating are applied, a curing time of 10 seconds to 30 minutes will generally suffice. Where a conventional oven is used, the total curing time preferably is at least 10 minutes, in particular at least 20 minutes, and at most 12 hours, in particular at most 6 hours. While longer curing times are not disadvantageous per se, it may be less attractive from an economical point of view. It is within the scope of a person skilled in the art to select suitable curing conditions. If so desired, curing can take place in one step or in multiple steps. Where more than one step is applied, the curing temperature of the second step will generally be higher than the curing temperature applied in the first step.

### The Filler

The starting material in the process of the present invention may or may not contain a filler.

Various types of fillers may be envisaged. In general, particulate, fibrous, and/or layered fillers may be used, of natural or synthetic origin. Combinations of various filler materials may be used. Fillers may be present in an amount of 10-95 wt. %, in particular in an amount of 20-80 wt. %, more in particular 40-70 wt. %, calculated on the total weight of the composite object.

Examples of suitable fillers are particulate materials. Within the context of the present specification, particulate materials are materials with an aspect ratio in the range of 10:1 to 1:1, preferably in the range of 8:1 to 1:1, more preferably in the range of 6:1 to 1:1. As used herein, “aspect ratio” is defined as the length of the particle, determined along its longest axis, over the largest diameter of the particle, determined along the axis that is perpendicular to the longest axis.

The particulate material may have a maximum length, determined along the longest axis of the particles in the material, of less than 20 mm, more preferably at most 15 mm, more preferably at most 10 mm, in particular at most 5 mm, in particular at most 2 mm. As a minimum value, an average length of the particles of 0.001 mm may be mentioned. In some embodiments, the average length of the particles is at least 0.05 mm, in particular at least 0.1 mm, more in particular at least 0.5 mm. In some embodiments, the average length of the particles is in the range of 0.5-5 mm, in particular 0.5-2 mm.

Suitable particulate material may, e.g., be in the form of powder, dust, pulp, broken fibers, flakes, or chips. Examples include wood chips, wood flakes, sawdust, hemp shives, (dried) grass, and pulp, e.g., pulp of (recycled) paper or other fiber pulp from sugar beets, fruits and vegetables, etc. Examples of plant-derived material that may be used as particulate material are cotton, flax, hemp, grass, reed, bamboo, coconut, miscanthus, coffee grounds, seed shells, e.g., from rice, burlap, kenaf, ramie, sisal, etc. and materials derived therefrom. In general plant material which has been comminuted to a suitable particle size, and where necessary dried to a suitable water content may be used.

The particulate material may comprise a natural material such as a material derived from plants or animals. Examples of plant-based materials include cellulose-based material such as fresh or used paper, fresh or used cardboard, wood or other plant material in any form, and combinations thereof. Cellulose-based materials may be derived from so-called virgin pulp which is obtained directly from the wood pulping process. This pulp can come from any plant material, but is mostly obtained from wood. Wood pulp comes from softwood trees such as spruce, pine, fir, larch, and hemlock and hardwood trees such as eucalyptus, popular, aspen, and birch. Additionally or alternatively, the cellulose-based material may comprise cellulose material derived from recycled paper, such as cellulose pulp obtained from regenerated books, papers, newspapers and periodicals, egg cartons, and other recycled paper or cardboard products. Combinations of cellulose sources may also be used. Other attractive sources of cellulose-based material are reject paper fiber, which is paper fiber that is too short to be suitable for use in the manufacture of paper, and any (mechanically and/or chemically) recycled material from any (composite) material, e.g. recycled furniture made from cellulose-based materials. In particular, (composite) materials made with the polymer mentioned here as a binder are attractive sources of the cellulose-based material. Use of these (recycled) materials is highly sustainable and low cost, allowing wide-spread use in, for example, furniture manufacturing.

Examples of animal-derived materials include feathers, down, hair and derivatives thereof such as wool, but also bone meal.

Further examples of suitable particulate materials include ceramic materials, including oxides, e.g. alumina, beryllia, ceria, zirconia, silica, titania, and mixtures and combinations thereof, and non-oxides such as carbide, boride, nitride, silicide, and mixtures and combi-nations thereof such as silicium carbide. For the purposes of the present specification glass is considered a ceramic material. Glass may, e.g., be used in the form of short fibers, glass beads, whether solid or hollow, and ground glass particles. Suitable particulate materials further include materials like micaceous fillers, calcium carbonate, and minerals such as phyllosilicates. Clay, sand, talcum, gypsum, etc. may also be used.

Suitable particulate materials also include polymer fillers, such as particles or short fibers of polyethylene, polypropylene, polystyrene, polyesters such as polyethylene terephthalate, polyvinylchloride, polyamide (e.g., nylon-6, nylon 6.6 etc.), polyacrylamide, and arylamide polymers such as aramid. Suitable particulate materials also include carbon fibers and carbon particulate materials. Comminuted cured polyester resin as used in the present invention may also be used as particulate material. Comminuted cured polyester resin containing a filler may also be used.

In some embodiments, particulate materials are used containing one or more organic particulate materials, e.g. selected from the group consisting of shives, wood dust, wood chips, and recycled paper. In other embodiments, the particulate material (also) contains one or more inorganic particulate materials, e.g. selected from the group consisting of (recycled) glass, stone, ceramic, minerals, and metals.

Suitable fillers also encompass fibrous materials. With the context of the present specification fibrous materials are materials with an aspect ratio of more than 10:1. Within the context of the present specification, the word “fiber” refers to monofilaments, multifilament yarns, threads, tapes, strips, and other elongate objects having a regular or irregular cross-section and a length substantially longer than the width and thickness.

Suitable fibrous material may, e.g., have a fiber length, determined over its longest axis, of at least 1 cm, preferably at least 3 cm, preferably at least 4 cm. For example, the fibrous material may have a fiber length, determined over its longest axis, of 1-20 cm. Preferably, the fibrous material has a fiber length of 1-10 cm. Long(er) fibers are preferred, because these provide strength to the composition.

The fibrous material may contain fibers having a diameter from 0.001 to 10 mm, preferably from 0.01 to 1 mm, more preferably from 10 to 500 μm. Thinner fibers are advantageous for many applications, as their use results in a smooth surface of the object. The fibers may, e.g., have an aspect ratio in the range of 20:1 to 200,000:1, preferably in the range of 200:1 to 20,000:1, more preferably in the range of 250:1 to 5000:1. The use of fibers with a relatively large aspect ratio makes for a combination of high strength and a smooth surface.

Fibers which may be used as fillers in the present invention may be oriented in a random (e.g., a non-woven sheet) or a non-random manner. The fibrous material is preferably non-woven sheet.

In the context of the present specification “oriented in a non-random manner” refers to all structures wherein fibers are oriented with respect to each other in an essentially regular manner. Examples of layers containing fibers oriented in a non-random manner include woven layers, knitted layers, layers wherein the fibers are oriented in parallel, and any other layers wherein fibers are connected to each other in a repeating patters.

Fiber orientation in the fibrous material may, for example, affect the strength of the end-product. Therefore, in some cases, it may be preferred to orientate the fibers in a manner that maximises the strength of the article. In some embodiments, at least 50% of the fibers are oriented in parallel, preferably at least 60% of the fibers are oriented in parallel, more preferably at least 70% of the fibers are oriented in parallel. In other cases, more anisotropic properties or bi-directional resistance may be required.

The fibrous material that may be used in the present invention may comprise plant-derived fibers, preferably cellulosic and/or lignocellulosic fibers. The fibrous material may also consist essentially of plant-derived fibers. Examples of fibers based on plant-derived fibers include flax, hemp, kenaf, jute, ramie, sisal, coconut, bamboo, and cotton. The fibrous material may also comprise an animal-derived fiber. The animal-derived fiber may be wool, hair, silk, and fibers derived from feathers (e.g., chicken feathers). Other parts of offal may also be used. The fibrous material may comprise synthetic fibers. Examples of suitable synthetic fibers are fibers derived from viscose, glass, polyesters, carbon, aramids, nylons, acrylics, poly-olefins and the like. The fibrous material may also be a mixture of fibers of different origin, such as a mixture of plant-derived fibers and synthetic fibers.

Within the context of the present specification, a composition a filler and a polymer also encompasses compositions in which the filler is provided in the form of thin layers stacked alternating with layers of polymer. Suitable layered materials generally comprise at least 2, in particular at least 4, up to 50, in particular up to 20 filler layers. The individual filler layers generally have a thickness of 0.1-10 mm, in particular 0.1-5 mm, more in particular 0.2-2 mm. The total thickness of the object may, e.g., be 0.5-200 mm. The polymer layers may have a thickness of, e.g., 10-4000 micron, in particular 10-2000 micron, more in particular 10-500 micron. Suitable fillers may, e.g., by wood (also indicated as wood veneer). Plywood is an example of this embodiment. Other layered fillers such as paper or cardboard may also be applied.

As will be understood by the skilled person, combinations of different types and materials may also be used as fillers.

The starting material used in the present invention may also be free from fillers. In that case, in one embodiment, it is a polymer foam.

In the starting material, the polymer has an extent of polymerisation of at least 0.7. In general, the starting material will be in the solid state at room temperature. It is a particular feature of the process of the invention that it allows processing of solid starting materials. The extent of polymerisation of the polymer in the starting material may be higher, e.g., at least 0.8, at least 0.9, or at least 0.95. It is a feature of the present invention that the process of the invention is also applicable to starting materials with a high extent of polymerisation. This is surprising because these materials are stable and resistant to degradation.

### Depolymerisation Step

In the process of the present invention a depolymerisation step is carried out in which the starting material is contacted contacting the starting material at a temperature of at least 60° C. with a nucleophile, the nucleophile comprising at least one of water, liquid polymer which is the polymerisation product of an aliphatic polyalcohol with 2-15 carbon atoms and an aliphatic polycarboxylic acid with 2-15 carbon atoms, and monomers of said polymer, to effect depolymerisation of the polymer and to reduce the extent of polymerisation of the polymer in the starting material with at least 0.1, to a value in the range of 0.1-0.8.

In the depolymerisation step a nucleophile is used, selected from water, liquid polymer which is the polymerisation product of an aliphatic polyalcohol with 2-15 carbon atoms and an aliphatic polycarboxylic acid with 2-15 carbon atoms, and monomers of said polymer. The selection of this specific group of nucleophiles ensures that no “new” compounds have to be added to the system.

The liquid polymer is of the same type as the polymer in the starting material, and the monomers used are of the same type as the monomers of the polymer in the starting material. It may be preferred for the liquid polymer to have the same chemical composition as the polymer in the starting material, and by the same token it may be preferred for the monomer mixture applied in the depolymerisation step to have the same composition as the monomers building up the polymer in the starting material. In the context of the present specification the wording “the same chemical composition” is defined as follows: two polymers have the same chemical compositions if they consist for at least 75% of the same monomers, in particular at least 80%, more in particular at least 90%.

The depolymerisation step is carried out at a temperature of at least 80° C. Higher temperatures will increase the reaction rate, but if the temperature becomes too high side reactions may start to occur. It may be preferred for the depolymerisation step to be carried out at a temperature of at least 90° C., in some embodiments at least 100° C. As a maximum temperature a value of 220° C. may be mentioned. Where the temperature if 100° C. is used, a pressure above atmospheric pressure may be applied. The preferred temperature range may also depend on the nucleophile that is used, on the desired reduction in extent of polymerisation, and upon the further conditions. These parameters will be discussed in more detail below.

If so desired, a catalyst may be present during the depolymerisation reaction. Suitable catalysts include the catalysts described above as suitable for use in polyester manufacture.

The depolymerisation step is generally carried out for a period of 1 minute to 24 hours, depending on temperature, pressure, the nature and amount of nucleophile, and the desired reduction in the extent of polymerisation. For example, at high temperature and superatmospheric pressure a time range of the order of minutes may suffice, while these conditions may require longer processing times. Depolymerisation times above 24 hours are less attractive from an economical point of view, and may additionally be associated with product degradation. It may be preferred for the depolymerisation step to be carried out for at for at most 12 hours, in particular at most 8 hours, more in particular at most 6 hours. In the depolymerisation step, the extent of polymerisation of the polymer is reduced with at least 0.1. The desired reduction of the extent of polymerisation in a particular case depends on the extent of polymerisation of the starting polymer and on the intended further processing of the polymer. Depending on the extent of polymerisation of the starting material and the desired product, the extent of polymerisation of the polymer may be reduced with at least 0.2, in particular at least 0.3, more in particular at least 0.4. The maximum reduction is 0.9.

The extent of polymerisation of the polymer after the depolymerisation step is in the range of 0.1-0.8.

It has been found not attractive to carry out the depolymerisation reaction to an extent of polymerisation of below 0.1. Not only does this require additional investments in time and energy, it also means that, in the case that the polymer is to be re-polymerised to form a new product additional water will have to be removed, as water will be generated in the reaction of an alcohol group with a carboxylic acid group. At the other end of the range, if the reduction of the extent of polymerisation is so limited that the end product still has an extent of polymerisation of 0.8, the effect of the process will generally be insufficient for the process to be commercially relevant. It may be preferred for the extent of polymerisation of the polymer after the depolymerisation step to be in the range of 0.2-0.8.

In one embodiment, the extent of polymerisation after the reaction is in the range of 0.1 to 0.6, in particular in the range of 0.2-0.5. This is the range where the polymer will generally be in the liquid phase (depending on the temperature), and that makes it possible to separate it from other components, e.g., filler material, to combine it with other materials, e.g., as an adhesive or a binder, or to reshape a material containing the polymer. These various aspects will be discussed below. In another embodiment, the extent of polymerisation of the product is in the range of 0.6-0.8. This range particularly attractive where reshaping of an existing filler-containing material is desired.

### Water as Nucleophile

In one embodiment, the nucleophile used in the present invention comprises water. Water may be present in the embodiment liquid polymer or monomers of said polymer are used as nucleophile. That embodiment will be discussed under the next heading. The present paragraphs are directed to the use of a nucleophile comprising water.

In the process of this embodiment, the starting material is treated with water at a temperature of at least 60° C. It is preferred for the temperature to be higher, to increase the depolymerisation rate. Accordingly, it is preferred for the process to be carried out at a temperature of at least 80° C., in particular at least 100° C. It has been found that the treatment with water at a temperature of at least 100° C. results in a fast depolymerisation, to a controlled extent. As a maximum, a value of 220° C. may be mentioned. Above that value side reactions may increase. Additionally, temperatures above that range are less attractive from an energetics point of view. It is preferred for the reaction temperature to be at least 110° C., in particular at least 120° C. It is also preferred for the temperature to be at most 200° C., in particular at most 180° C. In some embodiments, it may be preferred for the temperature to be at most 160° C. Where a temperature above 100° C. is used, it may be preferred for the pressure during the treatment with water to be above 1 bar. As a maximum, a value of 25 bars may be given. Above that value the process becomes less attractive from an economics point of view. It is particularly preferred for the pressure to be in the range of 1.5 to 15 bar, in particular in the range of 2-10 bar, still more in particular in the range of 3-8 bar.

In one embodiment, the reaction is carried out under autogenous pressure, i.e., the pressure is governed by the temperature and the amount of water in the reaction vessel.

Depending on the selected temperature and pressure, water will be present in the gaseous phase. In addition, depending on the amount of water, the temperature, and the pressure, liquid water may also be present. It has been found that the presence of liquid water may increase the depolymerisation rate. On the other hand, the presence of too much water may lead to an undesired extent of depolymerisation. Additionally, if it is intended to re-polymerise the depolymerised product, it may be attractive to limit the extent of depolymerisation through limiting the water content. The presence of excess water may also affect product homogeneity. Accordingly, it may be preferred to have an upper limit for the total amount of water present during the depolymerisation reaction. On the other hand, since the presence of water is required for an effective depolymerisation reaction, there is also a preference for a minimum amount.

In one embodiment, therefore, the amount of water provided for the depolymerisation reaction is in the range of 5 to 60 wt. %, in particular 5 to 40 wt. %, in some embodiments 10-30 wt. %, calculated on the amount of polymer provided to the depolymerisation reaction.

As a general remark, while it is possible to steer the extent of polymerisation of the final product through the amount of water, it may be more attractive to do so though the duration of the depolymerisation reaction.

Where the starting material contains a water-absorbing material, e.g., a porous hydrophilic filler, it may be desirable to add additional water to compensate for water that may be absorbed by the filler. An example may be the case that the starting material contains wood chips as filler.

### Liquid Polymer or Monomers of Said Polymer as Nucleophile

In one embodiment of the present invention, the starting material is contacted with a nucleophile comprising liquid polymer which is the polymerisation product of an aliphatic polyalcohol with 2-15 carbon atoms and an aliphatic polycarboxylic acid with 2-15 carbon atoms. Additionally or alternatively, the starting material is contacted with a nucleophile comprising monomers of said polymer, i.e., monomers selected from aliphatic polyalcohols with 2-15 carbon atoms and aliphatic polycarboxylic acids with 2-15 carbon atoms and combinations thereof. The preferences described above for the composition of the starting material also apply here, with the exception that the requirement for the amount of polyalcohol of at least three hydroxygroups and the amount of tricarboxylic acid are not required for the nucleating agent.

Where a nucleophile comprising one or more of polymer or monomers are used, the nucleophile will be a liquid medium. The temperature of the liquid medium will generally be in the range of 80-220° C., preferably in the range of 80-160° C.

The extent of polymerisation of the product obtained by this method will generally be between 0.1 and 0.7 in particular between 0.2 and 0.6, more in particular between 0.2 and 0.5.

Where liquid polymers or monomers thereof are used as nucleophile, some water may be present. In this embodiment the polymer to be depolymerised is in essence dissolved in liquid polymer/monomer. The amount of water in the liquid mixture is generally at most 40 wt. % of the total of the liquid mixture, in particular at most 30 wt. %, more in particular at most 20 wt. %. The presence of a low amount of water is preferred, because any water will have to be removed further on in the process.

In this embodiment, the amount of liquid medium should be sufficient to allow the polymer to disintegrate into the liquid medium. Therefore, in one embodiment the volume of the liquid medium is at least 50 vol. % of the volume of the polymer to be depolymerised therein. Because excess volumes are undesirable, it is preferred for the volume of the liquid medium to be at most 500 vol. % of the volume of the polymer to be depolymerised therein.

This embodiment is particularly attractive for depolymersiation of starting materials which consists for the most part, e.g., for at least 90 wt. % of the specified polyester polymer, in particular for at least 95 wt. %, more in particular at least 98 wt. % or at least 99 wt. %.

### Selection of the Starting Material and the Process

The process for treating a polymer-containing material according to the invention can be applied to starting materials containing a filler, but also in starting materials which do not contain a filler. Examples of various types of starting materials will be discussed below.

In one embodiment the starting material used in the process according to the invention is a polymer-containing material which consists for the most part, e.g., for at least 90 wt. % of the specified polyester polymer, in particular for at least 95 wt. %, more in particular at least 98 wt. % or at least 99 wt. %. In this case, the method of the invention may for example be used to convert the polymer into the liquid phase, generally to an extent of polymerisation in the range of 0.1 to 0.6 in particular or 0.2-0.5. Higher extents of polymerisation are also possible.

Where the starting material contains solid components, further indicated as fillers, there will be various options, depending on, among others, the nature of the filler, the relative amounts of polymer and filler, and the intended further use of the various compounds. In one embodiment, the polymer will be converted to the liquid phase, generally to an extent of polymerisation in the range of 0.1 to 0.6, preferably 0.2 to 0.5, and a separation step is carried out to separate liquid product polymer from the filler. The liquid product and the filler, which will generally still contain some polymer, will then be processed separately.

In another embodiment, the filler and the polymer will not be separated after the depolymerisation step. In this case the product of the depolymerisation step comprising polymer with a reduced extent of polymerisation and filler may be processed as such to form new objects. In this case, the desired extent of polymerisation after the depolymerisation step may be higher, e.g., at least 0.2, or at least 0.3 or at least 0.4. The general ranges provided above will still apply to this embodiment.

Depending on the nature of the starting material and the intended further processing, the starting material may be subjected to a size reduction step before being provided to the depolymerisation step. Where the material provided has a smaller size, the contact surface with water or steam will be larger, therewith increasing the reaction rate. On the other hand, especially where the filler is has a relatively large particle size or is fibrous in nature and its properties are to be re-used, it is important not to affect the properties of the filler by reducing the size of the particles to a too large extent. In other embodiments, size reduction will be carried out only to a limited extent, or will not be carried out at all, e.g., where the intention of the depolymerisation step is to be followed by a reshaping step. Size reduction after the depolymerisation step may also be attractive, because the depolymerisation of the polymer will soften the material, making size reduction easier to carry out. It is of course also possible to carry out size reduction between two depolymerisation steps.

In some embodiments, a shaping the step may be carried out. Within the context of the present specification, a shaping step is any step in which a material containing depolymerised polymer and filler is subjected to a step in which its shape is changed. This can be done, e.g., by bending, folding, flattening, or in any other way changing the shape of the object as a whole, but it can also be effected by combining materials and forming a new shape. Changing the shape of an existing object after depolymerisation may also be indicated herein as reshaping.

In some embodiments, an object comprising depolymerised polymer is subjected to a curing step to increase the extent of polymerisation, e.g., to a value of at least 0.7, at least 0.8, or at least 0.9. Immediately after curing, the water content of the object is generally below 10 wt. % (calculated on the total weight of the layered structure), preferably below 5 wt. %, more preferably below 2 wt. %, most preferably below 1 wt. %. Depending on the storage conditions, the water content of the object may increase after curing. For further information on the curing step reference is made to what is stated on curing in the context of the starting material.

In the following, various specific embodiments of the method according to the invention will be described, without the invention being limited thereto or thereby. Aspects of different embodiments may be combined, and further embodiments will be apparent to the skilled person.

### Processing of Filler-Free Polymer-Containing Material

In one embodiment, the polymer-containing material does not contain a filler. In this embodiment, the polymer-containing material will generally be a solid polymer material which consists for the most part, e.g., for at least 90 wt. % of polymer, in particular for at least 95 wt. % of polymer, for example in the form of a foam. In this case, the method of the invention will generally be used to convert the polymer into the liquid phase, generally to an extent of polymerisation in the range of 0.2 to 0.6, in particular 0.2 to 0.5.

It may be preferred that the solid polymer material not containing a filler is provided to the process according to the invention in a particulate form, e.g., in the form of particulates with a maximum particulate diameter of 10 cm (i.e. 90% of the particles has a diameter below this value). As compared to larger particulate material, the reduction in size leads to an increased reaction rate, because the contact area between the water and/or steam and the solid polymer material is increased. Additionally, the distance the steam has to travel to reach the core of the polymer-containing material is reduced. It may be preferred for the material to be in the form of particles with a maximum particulate diameter of 6 cm, in particular 4 cm, more in particular 2 cm, in some embodiments at most 5 mm. Milling may also be applied. Where the polymer-containing material does not contain a filler, it may be preferred to carry out the process in such a manner that the product of the depolymerisation reaction is a liquid product, to allow efficient removal from the depolymerisation reactor. In one embodiment, the extent of polymerisation after the depolymerisation step is in the range of 0.1 to 0.6, in particular 0.2-0.5, if it is desired to manufacture a liquid product. The more general ranges also apply to this embodiment. The use of a nucleophile comprising liquid polymer may be particularly preferred.

Where a liquid polymer is used as nucleophile it may be preferred to incorporate at least 40 wt. % of starting material into the nucleophile, to ensure an optimal use of reactor volume.

The product from the depolymerisation reaction may be processed as desired. It may optionally be subjected to one or more purification steps, e.g. a filtration step to remove remaining solid particulates, or a purification step using activated carbon to remove color-or odour-generating contaminants. Excess water may also be removed if so desired.

### Processing of Filler-Containing Polymer-Containing Material-Reshaping

In one embodiment, the process is intended to reshape an existing product. In this case, the starting material is a polymer-containing material containing polymer and a filler. The polymer-containing material generally contains 10-70 wt. % of polymer and 30-90 wt. % of a filler.

In this embodiment, a shaped object of a polymer-containing material is subjected to a depolymerisation step, followed by a shaping step and a curing step.

In this case, the extent of polymerisation after the depolymerisation reaction is generally above 0.2, in particular above 0.3, more in particular above 0.5.

After the depolymerisation reaction, the resulting product is flexible. The product is subjected to a force to change is shape, followed by a curing step. The curing step is generally carried out while keeping the product in its new shape, e.g., by using a mould or a press.

In this embodiment it is preferred for the nucleophile applied in the depolymerisation step to be steam.

### Processing of Filler-Containing Polymer-Containing Material-Separation of Polymer

In one embodiment, the process of the invention is intended to recover polymer from filler containing polymer-containing material. This process may be particularly attractive where the polymer-containing material has a high polymer content, and/or where polymer does not strongly adhere to the filler. For example, non-porous fillers such as glass particles or fibers, carbon particles or fibers, or polymer fillers such as fillers based on aramid are generally easier to separate from the polymer than porous natural fibers such as hemp fibers. Separability will also depend on the further properties of the filler.

In this embodiment, the process according to the invention comprises the steps of subjecting a filler-containing polymer-containing material to a depolymerisation step, followed by separating the depolymerised polymer from the filler.

In one embodiment, the extent of polymerisation after the reaction generally is in the range of 0.2 to 0.6, in particular in the range of 0.2-0.5. This is the range where the polymer will generally be in the liquid phase (depending on the temperature), and that makes it possible to separate it from the filler. The presence of liquid water will help to reduce the viscosity of the polymer medium, and this may improve the separation process.

The separation step can be carried out by methods known to the skilled person, e.g., through filtration or decantation. If so desired, pressing and/or washing may be applied to remove additional material from the filler. The polymer thus recovered can be used to manufacture new polymer-containing products. The filler from which the polymer has been separated can also be processed as desired.

### Creating New Particulate Starting Materials

In one embodiment, the process according to the invention is used to manufacture new particulate starting materials from existing products. In this embodiment, the process according to the invention comprises the steps of providing a filler-containing polymer-containing material in the form of particulates, and subjecting the material to a depolymerisation step, resulting in the formation of polymer-containing filler-containing particles.

In this embodiment, the extent of polymerisation after the depolymerisation reaction is generally above 0.3, in particular above 0.5. The polymer-containing material generally contains 10-70 wt. % of polymer and 30-90 wt. % of a filler. The use of water/steam as nucleophile is considered preferred in this embodiment. The particles can be re-used to manufacture new products, e.g. by combining them with one or more of additional polymer or additional filler material, and subjecting the mixture to a shaping step or curing step, e.g., as described in WO2022106724.

### Re-Use of the Product Obtained

The process according to the invention results in a polymer which is the polymerisation product of an aliphatic polyalcohol with 2-15 carbon atoms and an aliphatic polycarboxylic acid with 2-15 carbon atoms, which polymer has an extent of polymerisation of 0.1-0.8. The resulting product may or may not contain a filler, and depending on the extent of polymerisation and the presence or absence of a filler, may be in the liquid phase. The product of the process of the present invention can be used as a starting material in the manufacture of new products. They can, e.g., be combined with a filler. Suitable fillers are described above in the context of the starting materials. Suitable manufacturing and curing conditions are also described above in the context of the starting materials. Additionally, reference may be made to the products and processes described in WO2012052385, WO2012140238, WO2012140239, WO2012140237, WO2013121033, WO2020152082, WO2020212427, WO2021023495, WO2021105143, WO2022043330, and WO2022106724.

All percentages used herein are weight percentages, unless specified otherwise.

As will be evident to the skilled person, different embodiments of the present invention can be combined unless they are mutually exclusive. The headings used in the present specification are solely intended to improve readability, and have no legal consequences. Accordingly, embodiments discussed under different headings can be combined unless they are mutually exclusive.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

The following examples will illustrate the practice of the present invention in some of the preferred embodiments. The invention is not limited thereto or thereby.

### EXAMPLES

### Example 1: Depolymerisation and Reuse of Foam

A glycerol/citric acid (molar ratio 1:1) foam with an extent of polymerisation of above 0.9 and a density of about 250g/l was provided. The foam was provided in the form of chunks with a maximum diameter of 2 cm. The chunks were provided to an autoclave, where they were contacted with steam at a temperature of 150° C. (˜5 bars) for a period of 2 hours. During the depolymerisation reaction the foam depolymerised to form a liquid polymer with an extent of polymerisation of about 0.2. When the depolymerisation reaction was completed, the liquid polymer composition was removed from the reactor.

The liquid polymer (resin) could be used to make a new foam or be used as binder in the same way as a fresh resin without loss of functionality (see example 6).

### Example 2: Panel of Particulate Material From a Depolymerized Hemp-Fiber Compressed Panel

A compressed panel containing a non-woven hemp fiber mat and 50 wt. % of a citric acid/glycerol polymer (molar ratio 1:1) with an extent of polymerisation of above 0.8 was subjected to a size reduction step, resulting in the formation of particles with an average diameter of below 4 mm. The particles were brought into a depolymerisation reactor where they were contacted with steam at a temperature of 120° C. (˜2 bars) for a period of 2 hours.

The product of the depolymerisation step was a particulate material still containing the polymer. The polymer had an extent of polymerisation of about 0.3.

The resulting particles were dried for 2 hours at 85° C., and then compressed at a pressure of 15 bar and a temperature of 145° C. for a period of 10 minutes, to form a compressed panel of hemp-containing particles with a thickness of 8 mm. The panel was then subjected to curing for 2 hours at 160° C. The polymer in the compressed panel had an extent of polymerisation of at least 0.8. The resulting panel was smooth and had good physical properties, including a flexural strength of 25 MPa, which is well above the value for a commercially available particle board).

### Example 3: Re-Use of Depolymerized Hemp Fiber-Curved Panel as a Flat Hemp Fiber Panel

A compressed curved (90°) panel containing hemp fiber and 50 wt. % of a citric acid/glycerol polymer (molar ratio 1:1) with an extent of polymerisation of above 0.8 was provided. The plate material, which had previously been a chair, had a density of 1.1 g/ml.

The plate material was provided to a depolymerisation reactor, where it was contacted with steam at a temperature of ˜120° C. (˜2 bars). for a period of 4 hours. After the depolymerisation reaction had been completed, the plate material, which had been rigid before, was now flexible. The polymer had an extent of polymerisation of about 0.4. The plate material was dried for 3 hours at 80° C. It was then processed by flattening it in a press and cured at 145° C. and a pressure of 15 bars for two hours, to form a smooth rigid flat plate material with a polymer with an extent of polymerisation of above 0.8 and good mechanical properties.

### Example 4: Recycling of Glass-Fiber Panel

A composite panel containing a stack of 5 woven glass fiber mats and a citric acid/glycerol polymer (molar ratio 1:1) with an extent of polymerisation of above 0.9 was provided.

The panel was provided to a depolymerisation reactor, where it was contacted with water at a temperature of 150° C. for a period of 1 hour (˜6 bars). After the depolymerisation reaction had been completed, the polymer had an extent of polymerisation of about 0.3.

The liquid polymer was separated from the glass fiber mats by filtration, to recover liquid polymer and glass fiber. The recovered glass fiber mats (touch and visually the same as new mats) were re-used to make a new glass fiber composites panel, by combining them with the recovered resin (in an amount of 40-60 wt. %) and subjecting the composite to a curing step at 160° C. The cured polymer had an extent of polymerisation of above 0.8. Good mechanical properties were obtained, although the values were slightly decreased as compared to the original panel, with the tensile strength, flexural strength and elongation in flexion being of the order of 70% of the original values. Not wishing to be bound by theory it is believed that this may be due to the removal of sizing agent during the depolymerisation process. In any case, this example shows that it is possible to manufacture a panel with good properties from depolymerised resin and re-used glass fiber.

### Example 5: Depolymerisation of Foam in Liquid Resin and Reuse as Binder in Hemp Fiber Panel

A glycerol/citric acid (molar ratio 1:1) foam was provided with an extent of polymerisation of above 0.9 and a density of about 250 g/l. The foam was provided in the form of chunks with a maximum diameter of 2 cm.

The chunks were provided to a depolymerisation reactor, where they were contacted with a liquid glycerol/citric acid (molar ratio 1:1) polymer resin with an extent of polymerisation of about 0.5 and a water content of 10 wt. %. A mixture was prepared with 50/50 wt. % foam particles/liquid polymer resin and some additional water (equal to the amount of water in the liquid resin). The mixture was reacted for 4 hours under reflux and a reaction temperature of about 130° C. At the end of the reaction, a liquid polymer medium was obtained, with an estimated extent of polymerisation of about 0.5. It was found that further resin foam could easily be dissolved/depolymerised in the obtained liquid medium.

The thus obtained resin was used in the manufacture of a panel by impregnating a hemp mat with the resin followed by curing under pressure, in a method in accordance with WO2022106724. It was found that the properties of the panel were the same as the properties of a panel based on fresh resin.

### Example 6: Recycling of Particle Board Panel

A panel made of wood particles and a citric acid/glycerol polymer (molar ratio 1:1) with an extent of polymerisation of above 0.8 was provided (typical chipboard composition with small particles on the outside and larger particles on the inside and about 15 wt. % polymer overall).

The panel was provided to a depolymerisation reactor, where it was contacted with steam at a temperature of ˜120° C. (˜2 bars) for a period of 6 hours.

The panel was soft, swollen and the particles with the resin are loose. With a kitchen mixer the particles were further loosened and dried in an oven for 2 hours at 85° C. With a sieve the particles were separated in smaller and larger particles. A new panel was made from the recovered particles, by providing a layer of larger particles sandwiched between two layers of smaller particles, and subjecting the composite to curing under pressure at 145° C. and a pressure of 15 bars for 2 hours. This resulted in a new panel which was based completely on recycled material. The panel had a thickness of 2 mm. The panel had a smooth surface and good mechanical properties, as can be seen from a flexural strength of 11 MPa.

The experiment was repeated with the addition of a minor amount of additional polymer (5-20 wt. % calculated on the amount of polymer present in the panel before recycling). This resulted in a panel with a flexural strength of 14 MPa, which is the same as the flexural strength of the original panel. Not wishing to be bound by theory it is believed that during the depolymerisation process some resin may be absorbed by the wood particles, making less resin available for bonding the particles together. The addition of a limited amount of additional resin helps to provide additional bonding.

1. A process for treating a polymer-containing material, the process comprising the steps of
providing a starting material comprising a polymer which is a polyester derived from aliphatic polyalcohol with 2-15 carbon atoms, the aliphatic polyalcohol comprising at least 70 wt. % of polyalcohol with at least 3 hydroxyl groups, and aliphatic polycarboxylic acid with 2 to 15 carbon atoms, the aliphatic polycarboxylic acid comprising at least 70 wt. % of tricarboxylic acid, the polyester having an extent of polymerization, which is the ratio of the fraction of functional groups that have reacted to the maximum of those functional groups that can react, of at least 0.7, and
in a depolymerisation step contacting the starting material at a temperature of at least 80° C. for at most 24 hours with a nucleophile to effect depolymerisation of the polymer, resulting in a polymer with an extent of polymerisation which is reduced by at least 0.1 as compared to the extent of polymerisation of the polymer in the starting material, and is at a value in the range of 0.1-0.8,
wherein the nucleophile comprises at least one of
(i) water,
(ii) liquid polymer which is the polymerisation product of an aliphatic polyalcohol with 2-15 carbon atoms and an aliphatic polycarboxylic acid with 2-15 carbon atoms,
(iii) liquid monomers of said polymer, or
(iv) a combination thereof.

2. The process according to claim 1, wherein the starting material comprises a polymer which is a polyester derived from glycerol and citric acid.

3. The process according to claim 1, wherein the polyester in the starting material has an extent of polymerisation of at least 0.8.

4. The process according to claim 1, wherein when the nucleophile applied in the depolymerisation step comprises a liquid polymer, the liquid polymer has the same chemical composition as the polymer in the starting material, and when the nucleophile applied in the depolymerisation step comprises monomers, the monomer mixture has the same composition as the monomers building up the polymer in the starting material.

5. The process according to claim 1, wherein the depolymerisation step is carried out at a temperature of at least 90° C., at most 220° C., or a combination thereof.

6. The process according to claim 1, wherein the extent of polymerisation of the polymer after the depolymerisation step is in the range of 0.1 to 0.7.

7. The process according to claim 1, wherein the starting material comprises a filler.

8. The process according to claim 1, wherein before or after the depolymerisation step a size reduction step is carried out.

9. The process according to claim 7, wherein after the depolymerisation step a step is carried out in which liquid polymer produced in the depolymerisation step is separated from the filler.

10. The process according to claim 7, wherein after the depolymerisation step the combination of filler and polymer with a reduced extent of polymerisation is processed together.

11. The process according to claim 1, wherein an object comprising depolymerised polymer is subjected to a curing step to increase the extent of polymerisation.

12. The process according to claim 1, wherein the polymer-containing starting material does not contain a filler and the depolymerisation step is carried out to an extent of polymerisation in the range of 0.1 to 0.7.

13. The process according to claim 7, wherein a shaped object of a filler-containing polymer-containing material is subjected to a depolymerisation step, followed by a shaping step and a curing step.

14. The process of claim 1, wherein at least one of:
the polyester in the starting material has an extent of polymerisation of at least 0.9;
the extent of polymerisation of the polymer after the depolymerisation step is in the range of 0.2-0.6;
the depolymerisation step is carried out at a temperature of at least 100° C., at most 220° C., or a combination thereof;
an object comprising depolymerised polymer is subjected to a curing step to increase the extent of polymerisation to a value of at least 0.7;
the polymer-containing starting material does not contain a filler and the depolymerisation step is carried out to an extent of polymerisation in the range of 0.2 to 0.6; or a combination thereof.

15. The process of claim 1, wherein at least one of:
the polyester in the starting material has an extent of polymerisation of at least 0.95;
the extent of polymerisation of the polymer after the depolymerisation step is in the range of 0.2-0.5;
an object comprising depolymerised polymer is subjected to a curing step to increase the extent of polymerisation to a value of at least 0.8;
depolymerisation step is carried out to an extent of polymerisation in the range of 0.2-0.5; or
a combination thereof.

16. The process of claim 1, wherein an object comprising depolymerised polymer is subjected to a curing step to increase the extent of polymerisation to a value of at least 0.9.

17. The process of claim 12, wherein the nucleophile comprises the liquid polymer.

18. The process of claim 14, wherein the nucleophile comprises the liquid polymer.

19. The process of claim 15, wherein the nucleophile comprises the liquid polymer.
